(19) United States
(12) Patent Application Publication
(10) Pub. No.: US 2002/0040052 A1
(43) Pub. Date: Apr. 4, 2002

(54) Method for neurite outgrowth

(76) Inventors: Hisatomi Ito, Kobe (JP); Shinya Tamura, Kobe (JP); Toshitsugu Miyazaki, Kobe (JP)

Correspondence Address: AMIN & TUROCY, LLP, 1900 EAST 9TH STREET, NATIONAL CITY CENTER, 24TH FLOOR,, CLEVELAND, OH, 44114, US

(21) Appl. No.: 09/927,038

(22) Filed: Aug. 9, 2001

(30) Foreign Application Priority Data
Aug. 17, 2000 (JP) 2000-248021

(51) Int. Cl. A61K 31/353

(52) U.S. Cl. 514/456

(57) ABSTRACT

The present invention relates to methods for extending neurites, using a composition containing a polyalkoxyflavonoid having a specific structure, especially nobiletin or tangeretin. It is found that also a composition containing an extract from a plant belonging to the citrus family has an activity to extend neurites. These compositions are useful to prevent and/or improve or treat neurodegeneration diseases such as Alzheimer's dementia and encephalic ischemia by accelerating extension of neurites.

### BACKGROUND OF THE INVENTION

1. Field of the Invention

The present invention relates to methods for extending neurites of neurocytes and compositions having neurite extending effect. More specifically, the present invention relates to methods for preventing and/or improving or treating neurodegeneration diseases such as Alzheimer's dementia and cerebral ischemia by accelerating neurite extension, and compositions for extending neurites that are useful for these methods.

2. Description of the Related Art

With the shift to the aging society, the incidence of senile dementia has been increasing and this has become a serious social problem. Many diseases are known to cause senile dementia. Senile dementia is roughly classified into three types: dementia due to organic disorders of the brain; dementia associated with diseases of organs other than brain; and dementia due to physical diseases caused by stress. In particular, senile dementia is caused mostly by organic disorders of the brain, and the dementia of this type is further classified into two types, cerebrovascular dementia and Alzheimer's dementia, depending on its cause.

It is known today that cerebral vasodilators have some effect on cerebrovascular dementia. However, concerning Alzheimer's dementia, the cause of this disease is still unknown and there is no report on treatment methods or pharmacotherapy suitable to prevent its pathopoiesis as well as its advance. Therefore, there is a need to develop medicines that are effective with respect to the dementia caused by organic disorders of the brain, especially Alzheimer's dementia.

In recent years, neurotrophical factors secreted from neurocytes such as nerve growth factors (NGF) have been found to exhibit excellent effects on neurodegeneration diseases and have attracted public attention. An NGF is a factor that is important and necessary for nervous tissue to grow and maintain its function. An NGF is indispensable for the maturation, differentiation and viability of sensory nerves and sympathetic nerves in the peripheral nerves, and of magnocellular cholinergic neuron in the central nerves. An NGF also acts to prevent degeneration of neurocytes when the brain is damaged. In this regard, raising the NGF level in a living body seems to be effective as a treatment method for disorders of central function, such as Alzheimer's dementia and cerebrovascular dementia, spinal cord injuries, peripheral nerve injuries, diabetic neuropathy and disorder of peripheral function such as amyotrophic lateral sclerosis.

However, an NGF is a protein having a molecular weight of 13000 in the form of monomer and 26000 in the form of dimer, so that it cannot pass through the blood-brain barrier. Therefore, in order to treat disorders of central function, NGFs are required to be administrated intraventricularly. Moreover, it is difficult to prepare NGFs in large quantities. In these respects, there are many problems about the use of NGF itself. As a result, it is very difficult to use NGF itself clinically.

Y. Furukawa et al. disclose the use of catecholamines (epinephrine, norepinephrines) as an NGF synthesis accelerator (FEBS Lett., 208, 258 (1986) ). Further, it is disclosed that theanine (Japanese Laid-Open Patent Publication (Tokkai) No. 7-173059), eicosapentaenoic acid (EPA), and docosahexaenoic acid (DHA) (Japanese Laid-Open Patent Publication (Tokkai) No. 8-143454) serve as an NGF synthesis accelerator.

However, since epinephrine and norepinephrine are hormones, there is the problem that the quantitative balance of hormones in a living body may be lost if such a substance is administrated. It is another drawback that the above-described NGF synthesis accelerator may exhaust brain cells that are already in abnormal conditions because the NGF synthesis accelerator forcibly releases NGFs.

Therefore, in order to prevent and/or improve or treat senile dementia, low molecular weight substances that exhibit NGF-like activity appear to be effective.

On the other hand, with respect to polyalkoxyflavonoid, Japanese Laid-Open Patent Publication (Tokkai) No. 2000-80035 only describes that it has matrix metalloprotease inhibitory effect. Japanese Laid-Open Patent Publication (Tokkai) No.6-31627 has reported that alcoholic extracts of ginseng have an activating effect on neurocytes, but the substance that has the activating effect has not been specified.

### SUMMARY OF THE INVENTION

Therefore, with the foregoing in mind, it is an object of the present invention to provide a method for extending neurites of neurocytes without any side effects, and a method for preventing and/or treating neurodegeneration diseases using novel compositions having neurite extending effect.

The present invention provides a method for extending neurites including administering a composition to a subject, the composition including a polyalkoxyflavonoid represented by Formula 1, and a pharmaceutically acceptable carrier or a food material:

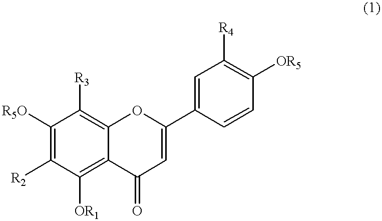

wherein R₁ is H or a lower alkyl group of C₁ to C₆; R₂, R₃ and R₄ are each independently H or an alkoxy group of C₁ to C₆; and R₅ is a lower alkyl group of C₁ to C₆.

The present invention also provides a method for extending neurites including administering a composition to a subject, the composition including an extract of a plant belonging to the citrus family, and a pharmaceutically acceptable carrier or food material.

The present invention also provides a method for preventing and/or treating neurodegeneration diseases including administering a composition to a subject, the composition including a polyalkoxyflavonoid represented by Formula 1, and a pharmaceutically acceptable carrier or food material:

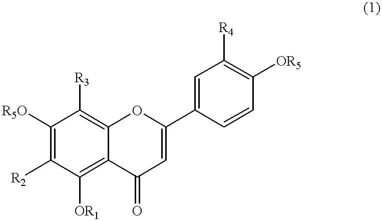

wherein R₁, R₂, R₃, R₄, and R₅ are the same as defined above.

The present invention also provides a method for preventing and/or treating neurodegeneration diseases including administering a composition to a subject, the composition including an extract of a plant belonging to the citrus family, and a pharmaceutically acceptable carrier or food material.

The present invention further provides a method for extending neurites including bringing a composition in contact with neurocytes, the composition including a polyalkoxyflavonoid represented by Formula 1 and a physiologically acceptable carrier:

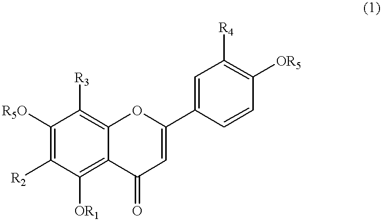

wherein R₁, R₂, R₃, R₄, and R₅ are the same as defined above.

The present invention further provides a method for extending neurites including bringing a composition in contact with neurocytes, the composition including an extract of a plant belonging to the citrus family and a physiologically acceptable carrier.

In a preferable embodiment, the above extract of a plant belonging to the citrus family contains a polyalkoxyflavonoid represented in Formula 1 in any of the methods.

In any of the above methods, in a preferable embodiment, the polyalkoxyflavonoid is nobiletin or tangeretin.

According to another aspect of the present invention, the present invention provides a composition that is a pharmaceutical composition or a quasi-drug composition for extending neurites or for preventing and/or treating neurodegeneration diseases and contains a polyalkoxyflavonoid represented by Formula 1 or an extract from a plant belonging to the citrus family and a pharmaceutically acceptable carrier:

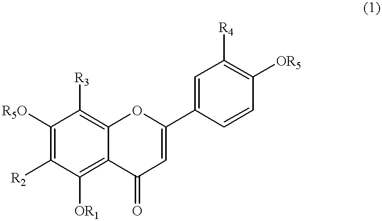

wherein R₁, R₂, R₃, R₄, and R₅ are the same as defined above.

The present invention also provides a composition that is a food composition for extending neurites or preventing and/or treating neurodegeneration diseases and contains a polyalkoxyflavonoid represented by Formula 1 or an extract from a plant belonging to the citrus family and a food material:

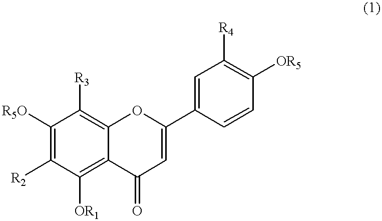

wherein R₁, R₂, R₃, R₄, and R₅ are the same as defined above.

The present invention further provides a composition that is a composition for cell treatment to extend neurites of neurocytes and contains a polyalkoxyflavonoid represented by Formula 1 or an extract from a plant belonging to the citrus family, and a physiologically acceptable carrier:

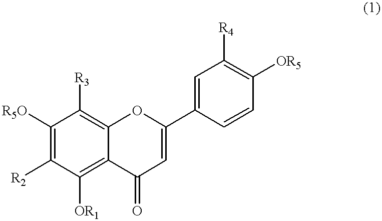

wherein R₁, R₂, R₃, R₄, and R₅ are the same as defined above.

In a preferable embodiment, the above extract from a plant belonging to the citrus family contains a polyalkoxyflavonoid represented by Formula 1 in any compositions.

In any of the above compositions, in a preferable embodiment, the polyalkoxyflavonoid is nobiletin or tangeretin.

According to the present invention, a composition that is highly safe and has excellent neurite extending effect on cells can be provided, and therefore, a method for extending neurites and a method for preventing and/or treating neurodegeneration diseases are provided. In particular, it is effective to use a composition containing nobiletin or tangeretin that is a polyalkoxyflavonoid as an active ingredient. The composition for extending neurites of the present invention can be used as a pharmaceutical, a quasi-drug or a food, and are effective to extend neurites and to prevent and/or treat neurodegeneration diseases such as Alzheimer's dementia and encephalic ischemia.

These and other advantages of the present invention will become apparent to those skilled in the art upon reading and understanding the following detailed description.

### DESCRIPTION OF THE PREFERRED EMBODIMENT

It is known that PC12 cells derived from adrenal medulla pheochromocytoma of rats extend neurites in response to NGFs. The inventors of the present invention examined various substances having NGF-like activities, using an evaluation system that utilizes these PC12 cells. As a result, the inventors of the present invention discovered that a polyalkoxyflavonoid having a specific chemical structure exhibits an excellent neurite extending effect.

In the present invention, “a composition for extending neurites” refers to a composition containing extracts of plants belonging to the citrus family or a composition containing a polyalkoxyflavonoid as represented by Formula 1:

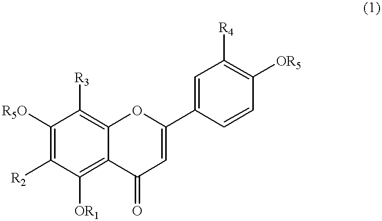

wherein R₁ is H or a lower alkyl group of C₁ to C₆ which may be branched; R₂, R₃ and R₄ are each independently H or an alkoxy group of C₁ to C₆ which may be branched; and R₅ is a lower alkyl group of C₁ to C₆ which may be branched. R₁ is preferably H or a lower alkyl group of C₁ to C₃. Preferably, R₂, R₃ and R₄ are each independently H or an alkoxy group of C₁ to C₃. R₅ is preferably a lower alkyl group of C₁ to C₃.

As examples of the polyalkoxyflavonoids represented by Formula 1, nobiletin represented by Formula 2 and tangeretin represented by Formula 3 are preferable because of the stability of these substances:

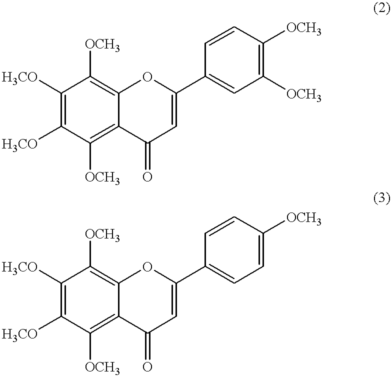

Nobiletin and tangeretin are contained in a large amount in plants belonging to the citrus family. Tangeretin is commercially available.

The polyalkoxyflavonoid represented by Formula 1 that is used in the present invention can be synthesized chemically, using methods well known to those skilled in the art. Alternatively, the polyalkoxyflavonoid can also be easily extracted from plants belonging to the citrus family as described later. In particular, a methoxyflavonoid such as nobiletin and tangeretin may be extracted and isolated from plants by the method as described by Jie Chem et al.(J. Agric. Food. Chem., 45, 364-368 (1997)).

The composition containing polyalkoxyflavonoid of the present invention includes pharmaceutical compositions, quasi-drug compositions, food compositions and compositions for cell treatment. Hereinafter, all of these may be simply referred to as “composition of the present invention”.

The minimum content of polyalkoxyflavonoid represented by Formula 1 in the composition of the present invention is preferably about 0.00001% by weight or more, more preferably about 0.0001% by weight or more. The maximum content of polyalkoxyflavonoid represented by Formula 1 in the composition of the present invention is preferably about 50% by weight or less, more preferably about 30% by weight or less. If the polyalkoxyflavonoid content is less than 0.00001% by weight, the neurite extending effect may not reach the desired level. On the other hand, if the content exceeds 50% by weight, better effects may not be expected. In the present invention, polyalkoxyflavonoids as described above can also be used in combination of two or more.

The composition of the present invention may also contain an extract from a plant belonging to the citrus family. “An extract from a plant belonging to the citrus family ” refers to an extract obtained from a plant belonging to the citrus family in the following manner. Preferably, the extract contains polyalkoxyflavonoid represented by Formula 1. More preferably, the extract from a plant belonging to the citrus family contains methoxyflavonoid. Most preferably, the extract from a plant belonging to the citrus family contains nobiletin represented by Formula 2 and/or tangeretin represented by Formula 3.

Examples of the plants belonging to the citrus family that are used for extraction include Citrus depressa, Citrus unshiu, Citrus tangerina, Citrus erythrosa, Citrus aurantium, Citrus natsudaidai, Citrus grandis, Citrus Junos, Citrus rericulata, Citrus lemon, Citrus trifoliata and Citrus medica L., all belonging to the citrus genus. In particular, Citrus depressa, Citrus unshiu and Citrus aurantium are preferable. In the present invention, the extracts from the above plants belonging to the citrus family may be used in combination of two or more.

The extracts from the above plants belonging to the citrus family may be obtained by extraction either from fresh plants or dried plants after collection. As for the parts to be used, fruits and peel of mature or immature plants, seeds, leaves, leafstalks, branches, roots and flowers of plants can be used. In particular, fruits and peel of mature or immature plants are preferable.

For example, extracts from the plants belonging to the citrus family can be obtained in the following manner.

First, a specified part of a plant belonging to the citrus family is immersed in an extractant. The amount of the extractant can be any amount as long as the plant is immersed in it, but amounts of twice to 100 times the weight of the plant belonging to the citrus family are preferable. There is no particular limitation regarding the extractant to be used. Examples of possible extractants to be used include lower alcohols such as methanol, ethanol, n-propanol, isopropanol and t-butanol; ketones such as acetone; esters such as ethylester acetate; ethers; halogenated hydrocarbon such as chloroform and dichloromethane; and water. These extractants can be used alone or in combination. In the present invention, methanol, ethanol, ethyl acetate, or combinations of these extractants with water are preferable. Considering the safety in a living body, ethanol or a mixed solvent of water and ethanol is more preferable because of their low toxicity. There is no particular limitation regarding the other conditions such as extracting temperatures, which can be set as appropriate by those skilled in the art.

The extract from a plant belonging to the citrus family obtained in this manner contains polyalkoxyflavonoid represented by Formula 1, preferably methoxyflavonoid, and most preferably nobiletin represented by Formula 2 and tangeretin represented by Formula 3. Furthermore, nobiletin and tangeretin can be isolated and purified from the extract of a plant belonging to the citrus family, for example, by column chromatography. The isolated substance can be identified as nobiletin and/or tangeretin by well-known means such as ¹H-NMR and ¹³C-NMR.

In the composition of the present invention, the content of the extract from a plant belonging to the citrus family is the same as in the composition comprising chemically synthesized polyalkoxyflavonoid as described above. Preferably, in the composition of the present invention, the extract from a plant belonging to the citrus family contains polyalkoxyflavonoid in a minimum amount of about 0.00001% by weight or more, more preferably about 0.0001% by weight or more. Preferably, in the composition of the present invention, the extract from a plant belonging to the citrus family contains polyalkoxyflavonoid in a maximum amount of about 30% by weight or less, more preferably about 15% by weight or less. If the polyalkoxyflavonoid content is less than 0.00001% by weight, the effect cannot appear sufficiently.

The composition of the present invention can be used either for oral administration or for parenteral administration.

The pharmaceutical composition of the present invention can contain pharmaceutically acceptable carriers that are commonly used for pharmaceutical production. The pharmaceutical composition can be made in any form, such as tablets, capsules, granules, syrup and injection. In the pharmaceutical composition, the content of polyalkoxyflavonoid represented by Formula 1 can be determined as appropriate by those skilled in the art.

Examples of the pharmaceutically acceptable carriers include excipients such as lactose, dextrin, sucrose, mannitol, cornstarch, and sorbitol, and adjuvants such as crystalline cellulose and polyvinylpyrrolidone. These can be used alone or in combination as appropriate. The pharmaceutical composition can be produced by a method suitable for the form of each pharmaceutical under Japanese Pharmacopeia and United States Pharmacopeia (USP). Furthermore, additives such as seasoning agents, coloring matters and sweetening agents can also be used, if necessary. The content of these additives can be selected as appropriate by those skilled in the art.

“Quasi-drug” refers to a product that is placed between drugs and cosmetics. More specifically, as is regulated in the Pharmaceutical Affairs Law of Japan, “quasi-drug” refers to a product that has a mild effect on the human body and is not an instrument or a tool, and similar products.

The quasi-drug composition of the present invention can contain pharmaceutically acceptable carriers that are commonly used for production of quasi-drugs. Furthermore, the quasi-drug composition of the present invention can contain other active ingredients such as vitamins. Additives such as sweetening agents, seasoning agents, coloring matters and antioxidants can also be used alone or in combination as appropriate. Examples of the forms of the quasi-drug composition include tablets, capsules, granules, jellies and drinkable preparations. In the quasi-drug composition, the content of polyalkoxyflavonoid represented by Formula 1 can be determined as appropriate by those skilled in the art. The quasi-drug composition of the present invention can be produced by a method well known to those skilled in the art.

The food composition of the present invention can be produced using various kinds of food ingredients as appropriate. Specific examples of food ingredients include lice, wheat, corn, potatoes, sweet potatoes, soybean meal, seaweed powder, starch syrup, lactose, glucose, fructose, sucrose and mannitol. These can be used alone or in combination as appropriate. There is no particular limitation regarding the form of the food composition of the present invention, and examples thereof are noodles, pasta, granules, tablets, jelly and liquid (drink). By using water or the like, if necessary, the food composition can be made into the desired form. Furthermore, seasoning agents, coloring matters, sweetening agents, edible oil, vitamins and the like can be added as appropriate. The content of polyalkoxyflavonoid represented by Formula 1 in the food composition can be determined as appropriate by those skilled in the art. The food composition can be produced by a method well known to those skilled in the art.

The composition for cell treatment of the present invention contains a physiologically acceptable carrier. Any physiologically acceptable carrier can be used as long as they are generally used to culture and grow cells, such as a culture medium. The content of polyalkoxyflavonoid represented by Formula 1 in the composition for cell treatment can be determined as appropriate by those skilled in the art. The composition for cell treatment can be produced by a method well known to those skilled in the art.

By using the compositions of the present invention obtained in the above-described manner, it is possible to extend neurites or prevent and/or treat neurodegeneration diseases.

More specifically, for example in vitro, by culturing cells in a medium containing the composition for cell treatment of the present invention, neurite extension of the cells can be observed. In vivo, by orally administrating the pharmaceutical composition of the present invention, neurite extension is accelerated, and furthermore, the prevention and/or treatment of neurodegeneration diseases such as Alzheimer's dementia and encephalic ischemia can be expected. The dose of the composition of the present invention, both in vitro and in vivo, can be determined as appropriate by those skilled in the art.

### EXAMPLES

Hereinafter, examples of the present invention will be described. The present invention is not limited by these examples.

### Example 1

First, 500 g of dried immature peel of Citrus unshiu were used for extraction with 90(v/v) % ethanol. An extract was filtered and concentrated under reduced pressure, and 28.2 g of a residue was obtained. The residue was separated with ethyl acetate-water, and then an ethyl acetate layer was concentrated under reduced pressure. Thus, 11.6 g of an extract of immature peel of Citrus unshiu was obtained.

The obtained extract of immature peel of Citrus unshiu was subjected to silica gel chromatography (eluent; ethyl acetate-n-hexane (1:1)), and fractionation was performed by HPLC (eluent: A 2% acetic acid aqueous solution, B. acetonitrile; A:B=85%:15% for 5 minutes, then gradient from A:B=85%:15% to A:B=40%:60% for 30 minutes; Flow rate: 10 ml/min.; Detection: UV 340nm). The fractions obtained at retention times of 28.5 minutes and 30.5 minutes were concentrated, dried, and crystallized with diethyl ether to obtain crystalline substances (1) and (2), respectively.

The melting point of the substance (1) was 137° C. to 138° C. The results of ¹³C-NMR and ¹H-NMR of the substance (1) were as follows:

NMR spectrum of the substance (1): ¹³C-NMR, δ (ppm), 55.6(OMe), 55.7(OMe), 61.4(OMe), 61.5(OMe), 61.8(OMe), 61.9(OMe), 106.3(CH), 108.9(CH), 111.8(CH), 114.3(C), 119.3(CH), 123.1(C), 137.7(C), 143.5(C), 147.5(C), 149.0(C), 150.9(C), 151.7(C), 160.7(C), 175.8(C═O); ¹H-NMR, δ (ppm), 3.77(s, 31), 3.83(s, 3H), 3.84(s, 3H), 3.87(s, 3H), 3.96(s, 3H), 4.01(s, 3H), 6.85(s, 1H), 7.15(d, J=8.6 Hz, 1H), 7.53(d, J-2.1 Hz, 1H), 7.64(dd, J=2.1, 8.6 Hz, 1H).

On the other hand, the melting point of the substance (2) was 156° C. to 157° C. The results of ¹³C-NMR and ¹H-NMR of the substance (2) were as follows:

NMR spectrum of the substance (2): ¹³C-NMR, δ (ppm), 55.5(OMe), 61.6(OMe), 61.8(OMe), 62.0(OMe), 62.2(OMe), 106.7(CH), 114.5(CH×2), 114.9(C), 123.8(CH×2), 138.1(C), 144.1(C), 147.7(C), 148.4(C), 151.3(C), 161.2(C), 162.3(C), 176.8(C═O); ¹H-NMR, δ (ppm), 3.88(s, 3H), 3.94(s, 3H), 4.02(s, 3H), 4.09(s, 3H), 6.59(s, 1H), 7.01 (d, J=8.8 Hz, 2H), 7.86(d, J=8.8 Hz, 2H).

Comparing the results of the measurement of the substances (1) and (2) with the values described in J. Agric. Food. Chem., 45, 364-368, (1997), the obtained substance (1) was identified as nobiletin and the substance (2) as tangeretin.

After the identification, the extract of immature peel of Citrus unshiu containing the above substances nobiletin and tangeretin was used without any further treatment as a test material A (composition for extending neurites).

Next, PC12 cells derived from adrenal medulla pheochromocytoma of rats were seeded in a serum-free DMEM/F12 medium containing 5 μg/ml of transferrin, 5 μg/ml of insulin and 20 nM of progesterone (GIBCO Corp., hereinafter, referred to as “DMEM-TIP medium”) at 2.0×10⁴ cells/well (flat-bottomed 24 well collagen coated plate, manufactured by IWAKI). Then, the cells were cultured overnight at 37° C. under 5% CO₂.

Thereafter, the PC12 cells were removed from the medium, transferred to the DEMEM-TIP medium containing 10 μg/ml of the above test material A and further cultured for 3 days.

After culturing for 3 days, for each well of the plate, microscopic observation was conducted with respect to the cells at 200 times magnification. The percentage of the cells with extended neurites (cells that have neurites longer than their diameter) to the total of more than 200 cells was calculated. The results are shown in Table 1.

### Example 2

The percentage of the cells with extended neurites was calculated in the same manner as in Example 1 except that PC12 cells were transferred to the DEMEM-TIP medium containing 50 μg/ml of the test material A. The results are shown in Table 1.

### Example 3

First, 500 g of dried peel of Citrus depressa were used for extraction with 90 (v/v) % ethanol. An extract was filtered and concentrated under reduced pressure, and 19.6 g of a residue was obtained. Then, the residue was separated with ethyl acetate-water, and an ethyl acetate layer was concentrated under reduced pressure. Thus, 8.5 g of an extract of Citrus depressa was obtained.

Analyzing the obtained extract of Citrus depressa by HPLC under the same conditions as in Example 1 using immature peel of Citrus unshiu, peaks corresponding to nobiletin (retention time: 28.2 minutes) and tangeretin (retention time: 30.5 minutes) were found.

The percentage of the cells with extended neurites was calculated in the same manner as in Example 1 except that the extract from Citrus depressa was used without any further treatment as a test material B instead of the test material A, and that PC12 cells were transferred to the DEMEM-TIP medium containing 10 μg/ml of the test material B. The results are shown in Table 1.

### Example 4

The percentage of the cells with extended neurites was calculated in the same manner as in Example 3 except that PC12 cells were transferred to the DEMEM-TIP medium containing 50 μg/ml of the test material B. The results are shown in Table 1.

### Example 5

First, 500 g of dried peel of Citrus aurantium were used for extraction with 90 (v/v) % ethanol. An extract was filtered and concentrated under reduced pressure, and 18.5 g of a residue were obtained. Then, the residue was separated with ethyl acetate-water, and then an ethyl acetate layer was concentrated under reduced pressure. Thus, 7.2 g of an extract of Citrus aurantium were obtained.

Analyzing the obtained extract of Citrus aurantium by HPLC under the same conditions as in Example 1 using immature peel of Citrus unshiu, peaks corresponding to nobiletin (retention time: 28.2 minutes) and tangeretin (retention time: 30.5 minutes) were found.

The percentage of the cells with extended neurites was calculated in the same manner as in Example 1 except that the extract from Citrus aurantium was used without any further treatment as a test material C instead of the test material A, and that PC12 cells were transferred to the DEMEM-TIP medium containing 10 μg/ml of the test material C. The results are shown in Table 1.

### Example 6

The percentage of the cells with extended neurites was calculated in the same manner as in Example 5 except that PC12 cells were transferred to the DEMEM-TIP medium containing 50 μg/ml of the test material C. The results are shown in Table 1.

### Example 7

The percentage of the cells with extended neurites was calculated in the same manner as in Example 1 except that nobiletin obtained in Example 1 was used without any further treatment as a test material D instead of the test material A and that PC12 cells were transferred to the DEMEM-TIP medium containing 10 μM of the test material D. The results are shown in Table 1.

### Example 8

The percentage of the cells with extended neurites was calculated in the same manner as in Example 1 except for using nobiletin obtained in Example 1 was used without any further treatment as a test material D instead of the test material A, and that PC12 cells were transferred to the DEMEM-TIP medium containing 50 μM of the test material D. The results are shown in Table 1.

### Example 9

The percentage of the cells with extended neurites was calculated in the same manner as in Example 1 except that nobiletin obtained in Example 1 was used without any further treatment as a test material D instead of the test material A, and that PC12 cells were transferred to the DEMEM-TIP medium containing 100 μM of the test material D. The results are shown in Table 1.

### Example 10

The percentage of the cells with extended neurites was calculated in the same manner as in Example 1 except that tangeretin obtained in Example 1 was used without any further treatment as a test material E instead of the test material A, and that PC12 cells were transferred to the DEMEM-TIP medium containing 10 μM of the test material E. The results are shown in Table 1.

### Example 11

The percentage of the cells with extended neurites was calculated in the same manner as in Example 1 except tangeretin obtained in Example 1 was used without any further treatment as a test material E instead of the test material A, and that PC12 cells were transferred to the DEMEM-TIP medium containing 100 μM of the test material E. The results are shown in Table 1.

### Comparative Example 1

The percentage of the cells with extended neurites was calculated in the same as in Example 1 except that PC12 cells were transferred to the DEMEM-TIP medium that did not contain the test material A of Example 1. The percentage in this case was used as a control. The results are shown in Table 1.

### Comparative Example 2

The percentage of the cells with extended neurites was calculated in the same manner as in Example 1 except that dibutyl cyclic (manufactured by Sigma Inc.) that has been reported to have neurite extending effect (Neurochem. Int. 33, 503, (1999)) was used without any further treatment as a test material F instead of the test material A, and that PC12 cells were transferred to the DEMEM-TIP medium containing 100 μM of the test material F. The results are shown in Table 1.

### Comparative Example 3

The percentage of the cells with extended neurites was calculated in the same manner as in Example 1 except that isobutylmethylxanthine (manufactured by Sigma Inc.) that has been reported to have neurite extending effect (J. Neurobiol. 19 (8), 681, (1988)) was used without any further treatment as a test material G instead of the test material A, and that PC12 cells were transferred to the DEMEM-TIP medium containing 100 μM of the test material G. The results are shown in Table 1.

TABLE 1

| | | | | Relative value |
|---|---|---|---|---|
| | | Concentration of | Ratio of | of the ratio of |
| | | a composition | the cells | the cells with |
| | | for extending | with ex- | extended |
| | Active ingredient | neurites of the | tended | neurites to |
| | in the test | present invention | neurites | control |
| | material | in the medium | (%) | (Com. Ex. 1)¹⁾ |
| Ex. 1 | extract of | 10 μg/ml | 10.2 | 2.9 |
| | immature peel of | | | |
| | Citrus unshiu | | | |
| Ex. 2 | extract of | 50 μg/ml | 18.6 | 5.3 |
| | immature peel of | | | |
| | Citrus unshiu | | | |
| Ex. 3 | extract of Citrus | 10 μg/ml | 7.5 | 2.1 |
| | depressa | | | |
| Ex. 4 | extract of Citrus | 50 μg/ml | 16.8 | 4.8 |
| | depressa | | | |
| Ex. 5 | extract of Citrus | 10 μg/ml | 7.1 | 2.0 |
| | aurantium | | | |
| Ex. 6 | extract of Citrus | 50 μg/ml | 15.9 | 4.5 |
| | aurantium | | | |
| Ex. 7 | nobiletin | 10 μM | 8.7 | 2.5 |
| Ex. 8 | nobiletin | 50 μM | 16.6 | 4.7 |
| Ex. 9 | nobiletin | 100 μM | 42.8 | 12.2 |
| Ex. 10 | tangeretin | 10 μM | 4.8 | 1.4 |
| Ex. 11 | tangeretin | 100 μM | 12.2 | 3.5 |
| Com. | none (control) | — | 3.5 | 1.0 |
| Ex. 1 | | | | |
| Com. | dibutyl cyclic | 100 μM | 11.6 | 3.3 |
| Ex. 2 | AMP | | | |
| Com. | Isobutylmethyl- | 100 μM | 11.8 | 3.4 |
| Ex. 3 | xanthine | | | |

¹⁾Relative value is the value obtained by dividing “the percentage of the cells with extended neurites” by the control value (Comparative Example 1).

As shown in Table 1, in comparison with the control of Comparative Example 1, all of the test materials A to E used in Examples 1 to 11 have excellent neurite extending effect to cells. According to the results of Examples 1 to 11, the higher concentration the test materials that are added to the cells have, the greater the neurite extending effect is. These values are equivalent or more than the results of test materials F and G known to have neurite extending activity in Comparative Examples 2 and 3. From this regard, it is evident that all of the test materials A to E used in Examples 1 to 11 are useful as compositions for extending neurites.

### Example 12

### Production of Food Products

Using the test material A (an extract of immature peel of Citrus unshiu) obtained in Example 1, food products having the composition shown in Table 2 below were prepared.

TABLE 2

| Component | Weight (kg) |
|---|---|
| extract of immature peel of Citrus | 2.0 |
| depressa | |
| soybean saponin | 2.0 |
| black vinegar extract | 2.0 |
| apple fiber | 2.0 |
| lecithin | 1.0 |
| fructo-oligosaccharide | 2.0 |
| fructose | 1.0 |
| powdered vinegar | 0.1 |
| cyclodextrin | 1.0 |
| honey | 1.0 |
| bone dust | 1.0 |
| dextrin | 4.9 |

The components were mixed in granulator, and granulated with spraying water. Then, granules obtained were dried at blowing temperature of 80° C.

### Example 13

### Production of Hard Gelatin Capsules

Using the test material C (an extract of Citrus depressa) obtained in Example 3, hard gelatin capsules having the composition shown in Table 3 below were prepared.

TABLE 3

| | |
|---|---|
| Component | Quantity (mg/capsule) |

| | | |
|---|---|---|
| extract of Citrus depressa | 250 | |
| starch | 100 | |
| cellulose | 100 | |
| magnesium stearate | 10 | |
| total | 460 | mg |

### Example 14

### Production of Tablets

Using the test material D (nobiletin) described in Example 7, tablets having the composition shown in Table 4 below were prepared.

TABLE 4

| | |
|---|---|
| Component | Quantity (mg/capsule) |

| | | |
|---|---|---|
| nobiletin | 250 | |
| cellulose | 400 | |
| silicon dioxide | 10 | |
| magnesium stearate | 5 | |
| total | 665 | mg |

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. A method for extending neurites comprising administering a composition to a subject, the composition comprising a polyalkoxyflavonoid represented by Formula 1, and a pharmaceutically acceptable carrier or a food material: 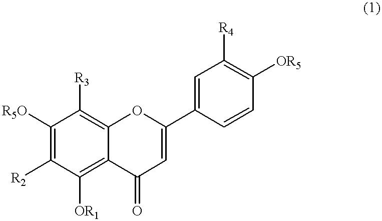
wherein R₁ is H or a lower alkyl group of C₁ to C₆; R₂, R₃ and R₄ are each independently H or an alkoxy group of C₁ to C₆; and R₅ is a lower alkyl group of C₁ to C₆.

2. The method of claim 1, wherein the polyalkoxyflavonoid is nobiletin or tangeretin.

3. A method for extending neurites comprising administering a composition to a subject, the composition comprising an extract from a plant belonging to the citrus family, and a pharmaceutically acceptable carrier or a food material.

4. The method of claim 3, wherein the extract from a plant belonging to the citrus family comprises a polyalkoxyflavonoid represented by Formula 1: 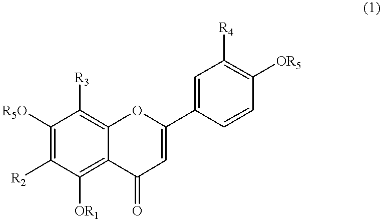
wherein R₁ is H or a lower alkyl group of C₁ to C₆; R₂, R₃ and R₄ are each independently H or an alkoxy group of C₁ to C₆; and R₅ is a lower alkyl group of C₁ to C₆.

5. The method of claim 4, wherein the polyalkoxyflavonoid is nobiletin or tangeretin.

6. A method for preventing and/or treating neurodegeneration diseases comprising administering a composition to a subject, the composition comprising a polyalkoxyflavonoid represented by Formula 1, and a pharmaceutically acceptable carrier or a food material: 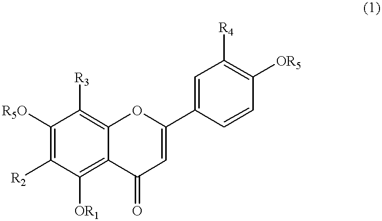
wherein R₁ is H or a lower alkyl group of C₁ to C₆; R₂, R₃ and R₄ are each independently H or an alkoxy group of C₁ to C₆; and R₅ is a lower alkyl group of C₁ to C₆.

7. The method of claim 6, wherein the polyalkoxyflavonoid is nobiletin or tangeretin.

8. A method for preventing and/or treating neurodegeneration diseases comprising administering a composition to a subject, the composition comprising an extract from a plant belonging to the citrus family, and a pharmaceutically acceptable carrier or a food material.

9. The method of claim 8, wherein the extract from a plant belonging to the citrus family comprises a polyalkoxyflavonoid represented by Formula 1: 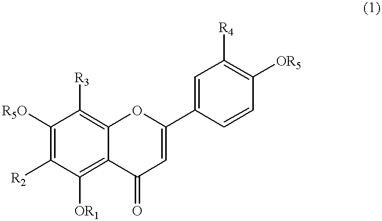
wherein R₁ is H or a lower alkyl group of C₁ to C₆; R₂, R₃ and R₄ are each independently H or an alkoxy group of C₁ to C₆; and R₅ is a lower alkyl group of C₁ to C₆.

10. The method of claim 9, wherein the polyalkoxyflavonoid is nobiletin or tangeretin.

11. A method for extending neurites comprising bringing a composition in contact with neurocytes, the composition comprising a polyalkoxyflavonoid represented by Formula 1, and a physiologically acceptable carrier: 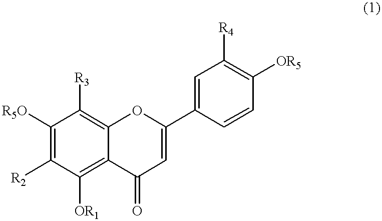
wherein R₁ is H or a lower alkyl group of C₁ to C₆; R₂, R₃ and R₄ are each independently H or an alkoxy group of C₁ to C₆; and R₅ is a lower alkyl group of C₁ to C₆.

12. The method of claim 11, wherein the polyalkoxyflavonoid is nobiletin or tangeretin.

13. A method for extending neurites comprising bringing a composition in contact with neurocytes, the composition comprising an extract from a plant belonging to the citrus family, and a physiologically acceptable carrier.

14. The method of claim 13, wherein the extract from a plant belonging to the citrus family comprises polyalkoxyflavonoid represented by Formula 1: 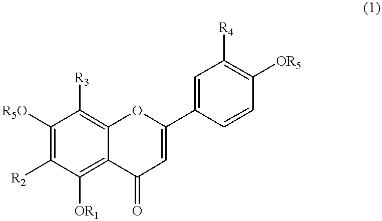
wherein R₁ is H or a lower alkyl group of C₁ to C₆; R₂, R₃ and R₄ are each independently H or an alkoxy group of C₁ to C₆; and R₅ is a lower alkyl group of C₁ to C6.

15. The method of claim 14, wherein the polyalkoxyflavonoid is nobiletin or tangeretin.
